(19) United States
(12) Patent Application Publication
(10) Pub. No.: US 2021/0340693 A1
(43) Pub. Date: Nov. 4, 2021

(54) FIBER AND METHOD FOR PREPARING THE SAME AND ARTIFICIAL LIGAMENT/TENDON

(71) Applicant: INDUSTRIAL TECHNOLOGY RESEARCH INSTITUTE, Hsinchu (TW)

(72) Inventors: Shinn-Jen CHANG, Hsinchu City (TW); Wesley Jen-Yang CHANG, Tainan City (TW); Hsin-Hsin SHEN, Zhudong Township (TW); Pei-Yi TSAI, Hsinchu City (TW)

(73) Assignee: INDUSTRIAL TECHNOLOGY RESEARCH INSTITUTE, Hsinchu (TW)

(21) Appl. No.: 17/244,742

(22) Filed: Apr. 29, 2021

Related U.S. Application Data
(60) Provisional application No. 63/017,113, filed on Apr. 29, 2020.

(30) Foreign Application Priority Data
Apr. 29, 2020 (TW) 109114303

(51) Int. Cl. D01F 8/14; A61F 2/08; D01F 1/10; D01F 8/18; D02G 3/04; B29C 48/05

(52) U.S. Cl. CPC D01F 8/14; A61F 2/08; D01F 1/10; D01F 8/18; D02G 3/04; B29C 48/05; B29C 48/505; D10B 2101/08; D10B 2509/00; D10B 2401/061; A61F 2210/00; A61F 2230/0069; D10B 2331/04

(57) ABSTRACT

A method of preparing fiber includes blending bio-compatible ceramic powder and first polyester to form a ceramic powder composition, wherein the bio-compatible ceramic powder and the first polyester have a weight ratio of 10:90 to 60:40. The method further includes blending the ceramic powder composition and second polyester to form a composite material, wherein the ceramic powder composition and the second polyester have a weight ratio of 0.4:99.6 to 40:60. The method also spins the composite material to form a fiber. The first polyester has an intrinsic viscosity (IV) of 0.35 dL/g to 0.55 dL/g, and the second polyester has an intrinsic viscosity (IV) of 0.6 dL/g to 0.8 g/dL. The fiber can be woven to form an artificial ligament/tendon.

### CROSS REFERENCE TO RELATED APPLICATIONS

This application claims the benefit of U.S. Provisional Application No. 63/017,113, filed on Apr. 29, 2020, the entirety of which is/are incorporated by reference herein.

The present application is based on, and claims priority from, Taiwan Application Serial Number 109114303, filed on Apr. 29, 2020, the disclosure of which is hereby incorporated by reference herein in its entirety

### TECHNICAL FIELD

The technical field relates to a fiber, a method for preparing the same and an artificial ligament/tendon.

### BACKGROUND

Clinical operations often use autologous ligaments/tendons and artificial ligaments/tendons for medical treatment. However, autologous tissue repair has its inconveniences and negative effects on patients. Commercially available artificial ligament/tendon implants will cause poor histocompatibility, inflammation, and swelling after long-term use, which cannot effectively promote the regeneration and integration of autologous tissues, and may even wear, loosen, and break. Whether in clinic or in the market, there is an urgent need for tissue-compatible artificial ligament/tendon materials to overcome the problem of tissue regeneration and repair. The conventional skill often forms a coating of bio-compatible ceramic powder on the fiber by dip coating to make the artificial fiber have excellent bio-compatibility. However, the bio-compatible ceramic powder cannot be evenly dispersed into the fiber using this method, and the coating may easily peel, lowering the bio-compatibility. Moreover, the peeled fragment may cause side effects such as inflammation. For evenly dispersing the bio-compatible ceramic powder into the fiber, a dispersant is usually added, lowering the interfacial energy between the ceramic powder and the carrier resin. However, the commercially available dispersant not only easily migrates to the surface of the fiber and causes cytotoxicity due to its low molecular weight and many active functional groups, but it also violates medical regulations. In other words, the common small molecular dispersant cannot be used in the composite material of the bio-compatible ceramic powder and the carrier resin.

Accordingly, a novel method for dispersing the bio-compatible ceramic powder in the carrier resin, spinning the composite to form a fiber, and weaving the fiber to form an artificial ligament/tendon to meet the clinical or market demand is called for.

### SUMMARY

One embodiment of the disclosure provides a fiber, including: 0.5 to 4 parts by weight of a bio-compatible ceramic powder region; and 96 to 99.5 parts by weight of a polyester region, wherein the bio-compatible ceramic powder region is distributed in the polyester region, at least 90% of the bio-compatible ceramic powder region has a diameter of less than or equal to 300 nm and greater than 10 nm, and the cell viability of bio-toxicity test of the fiber is higher than 70%.

In some embodiments, the fiber has a diameter of 2 micrometers to 150 micrometers.

In some embodiments, the polyester region includes polyethylene terephthalate, polybutylene terephthalate, or a combination thereof, and the bio-compatible ceramic powder region includes hydroxyapatite, tricalcium phosphate, calcium sulfate, or a combination thereof.

In some embodiments, the fiber is free of dispersant.

In some embodiments, the fiber has a cell viability of bio-toxicity test higher than 100%.

On embodiment of the disclosure provides an artificial ligament/tendon being woven from the described fiber.

One embodiment provides a method of preparing a fiber, including: blending bio-compatible ceramic powder and first polyester to form a ceramic powder composition, and the bio-compatible ceramic powder and the first polyester have a weight ratio of 10:90 to 60:40; blending the ceramic powder composition and second polyester to form a composite material, wherein the ceramic powder composition and the second polyester have a weight ratio of 0.4:99.6 to 40:60; and spinning the composite material to form a fiber, wherein the first polyester has an intrinsic viscosity (IV) of 0.35 dL/g to 0.55 dL/g, and the second polyester has an intrinsic viscosity (IV) of 0.6 dL/g to 0.8 g/dL.

In some embodiments, the fiber includes: 0.5 to 4 parts by weight of a bio-compatible ceramic powder region; and 96 to 99.5 parts by weight of a polyester region, wherein the bio-compatible ceramic powder region is distributed in the polyester region, at least 90% of the bio-compatible ceramic powder region has a diameter of less than or equal to 300 nm and greater than 10 nm, and the cell viability of bio-toxicity test of the fiber is higher than 70%.

In some embodiments, the fiber has a diameter of 2 micrometers to 150 micrometers.

In some embodiments, the first polyester and the second polyester include polyethylene terephthalate, polybutylene terephthalate, or a combination thereof, and the bio-compatible ceramic powder includes hydroxyapatite, tricalcium phosphate, calcium sulfate, or a combination thereof.

In some embodiments, the fiber is free of dispersant.

In some embodiments, the intrinsic viscosity difference (ΔIV) between the first polyester and the second polyester is greater than or equal to 0.1 dL/g and less than or equal to 0.45 dL/g.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment provides a method of preparing a fiber. First, bio-compatible ceramic powder and first polyester are blended to form a ceramic powder composition. It should be understood that the method of blending the bio-compatible ceramic powder and the first polyester can be any suitable blending method known in the art, such as melt blending. In one embodiment, the bio-compatible ceramic powder includes hydroxyapatite, tricalcium phosphate, calcium sulfate, or a combination thereof, and its average diameter is 20 nanometers to 100 nanometers (or 40 nanometers to 80 nanometers). If the diameter of the bio-compatible ceramic powder is too large, the filament will be easily broken during the spinning or the fiber product will easily break. The first polyester can be polyethylene terephthalate, polybutylene terephthalate, or a combination thereof, and the first polyester has an intrinsic viscosity (IV) of 0.35 dL/g to 0.55 dL/g (or 0.4 dL/g to 0.55 dL/g). If the intrinsic viscosity of the first polyester is too low, the mechanical strength of the fiber product will be affected. If the intrinsic viscosity of the first polyester is too high, the bio-compatible ceramic powder will aggregate and cannot be efficiently dispersed in the first polyester, and the diameter of the bio-compatible ceramic powder region in the final product will be too large. In some embodiment, the bio-compatible ceramic powder and the first polyester have a weight ratio of 10:90 to 60:40 (or 20:80 to 60:40). If the bio-compatible ceramic powder amount is too low, the bio-compatibility of the ceramic powder composition and the fiber will be insufficient. If the bio-compatible ceramic powder amount is too high, the bio-compatible ceramic powder will aggregate and cannot be efficiently dispersed in the first polyester, and the diameter of the bio-compatible ceramic powder region in the final product will be too large. As such, the filament will be easily broken during the spinning or the fiber product will easily break.

Subsequently, the ceramic powder composition and second polyester are blended to form a composite material. It should be understood that the method of blending the ceramic powder composition and the second polyester can be any suitable blending method known in the art, such as melt blending. In some embodiments, the second polyester can be polyethylene terephthalate, polybutylene terephthalate, or a combination thereof, and the second polyester has an intrinsic viscosity (IV) of 0.6 dL/g to 0.8 dL/g (or 0.6 dL/g to 0.7 dL/g). If the intrinsic viscosity of the second polyester is too low, the mechanical strength of the fiber product will be affected. If the intrinsic viscosity of the second polyester is too high, the spinning will be difficult. In some embodiments, the intrinsic viscosity difference (ΔIV) between the first polyester and the second polyester is greater than or equal to 0.1 dL/g and less than or equal to 0.45 dL/g. Note that the first polyester and the second polyester should be same type polyester, e.g. both are polyethylene terephthalate. If the first polyester and the second polyester are different types, the ceramic powder composition may not be efficiently dispersed in the second polyester. In some embodiments, the ceramic powder composition and the second polyester have a weight ratio of 0.4:99.6 to 40:60. If the ceramic powder composition amount is too low, the bio-compatibility of the ceramic powder composition and the fiber will be insufficient. If the ceramic powder composition amount is too high, the filament will be easily broken during the spinning or the fiber product will easily break.

Note that if the first polyester, the second polyester, and the bio-compatible ceramic powder are simultaneously blended, the bio-compatible ceramic powder will aggregate and cannot be efficiently dispersed. Similarly, if the first polyester and the second polyester are firstly blended, and the bio-compatible ceramic powder is then added to blend, the bio-compatible ceramic powder will aggregate and cannot be efficiently dispersed.

Subsequently, the composite material is spun to form a fiber. It should be understood that the method of spinning the composite material can be any suitable spinning method known in the art, such as melt spinning. In some embodiments, the fiber includes 0.5 to 4 parts by weight of a bio-compatible ceramic powder region and 96 to 99.5 parts by weight of a polyester region. In some embodiments, the fiber includes 0.5 to 3 parts by weight of a bio-compatible ceramic powder region and 97 to 99.5 parts by weight of a polyester region. The bio-compatible ceramic powder region is distributed in the polyester region. It should be understood that the bio-compatible ceramic powder region comes from the bio-compatible ceramic powder in the composite material, and the polyester region comes from the first polyester and the second polyester in the composite material. In some embodiments, the bio-compatible ceramic powder region is the region that the bio-compatible ceramic powder aggregates, and the polyester region is the region excluding the bio-compatible ceramic powder. At least 90% of the bio-compatible ceramic powder region has a diameter of less than or equal to 300 nm and greater than 10 nm. If the diameter of the bio-compatible ceramic powder region is too large, the composite material will easily block the spinning nozzle and break the filament, and the fiber product will have a low mechanical strength. The cell viability of bio-toxicity test of the composite material before spinning is higher than 70%, which means that the composite material is non-cytotoxic. The cell viability of bio-toxicity test of the fiber after spinning is higher than 70% or even higher than 100%, which means that the fiber in some embodiments not only be non-cytotoxic but also promotes cell growth.

In one embodiment, the fiber has a diameter of 2 micrometers to 150 micrometers, or 10 micrometers to 110 micrometers. In some embodiments, the fiber has a diameter of 10 micrometers to 60 micrometers. In some embodiments, the fiber containing the bio-compatible ceramic powder region and the polyester region is free of an additional dispersant, such as a dispersant having a molecular weight of less than or equal to 5000 and greater than 0, or a dispersant having a molecular weight of less than or equal to 3000 and greater than 0. Because the general dispersant easily migrates to the surface of the fiber and has cytotoxicity, which is not suitable to be applied to medical materials such as the artificial ligament/tendon.

In one embodiment, the fiber can be woven to form artificial ligament/tendon. It should be understood that the method of weaving the fiber can be any suitable weaving method known in the art. Because the fiber in the embodiments of the disclosure may promote bone cell differentiation, it is more suitable for artificial ligament/tendon than the fiber prepared from the common bio-compatible material. As proven by clinical animal experiments, the fiber of the disclosure can be woven to form an artificial ligament, and the artificial ligament can be implanted into animals without inducing liver and kidney toxicity (e.g. bio-compatible). The surrounding soft tissues successfully grow into the artificial ligament as a ligamentation phenomenon. The gap decreases between the ligament and the bone, and healing phenomenon was observed between the bone screw and the bone tunnel. In addition, the artificial ligament of the disclosure has a higher ultimate tensile strength than the commercially available artificial ligament after being implanted into animal in one month.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

The intrinsic viscosities (IV) of the polyesters in following Examples were measured according ASTM D4603.

### Example 1

194.18 parts by weight of dimethyl terephthalate, 173.79 parts by weight of ethylene glycol, and 0.01 parts by weight of tetrabutyl titanate were reacted at about 200° C. for about 2 hours, and then heated to about 260° C. and vacuumed to a pressured of about 4 torr to react for about 1 hour, and then heated to about 270° C. and vacuumed to a pressured of about 0.1 torr to react until its intrinsic viscosity achieving 0.433 dL/g. The product such as the polyethylene terephthalate (PET, intrinsic viscosity was 0.433 dL/g) serving as first polyester was put into a vacuum oven, heated to about 120° C. and vacuumed to remove water. Hydroxyapatite powder (original average diameter was about 60 nm, commercially available from KING MEITEK INDUSTRIAL CO., LTD.) served as bio-compatible ceramic powder. 60 parts by weight of the anhydrous PET and 40 parts by weight of hydroxyapatite powder were fed into a twin screw extruder, and then melt blended and dispersed at a screw temperature of about 265° C. and a rotating speed of 40 rpm to prepare ceramic powder composition. The cytotoxicity of the ceramic powder composition was measured according to the standard ISO10993-1 (MTT assay), and its cell viability was ≥70% (non-cytotoxicity).

### Example 2

Example 2 was similar to Example 1, and the difference in Example 2 was the weight ratio of the first polyester and the hydroxyapatite being changed from 60:40 to 40:60. The other processes and method of measuring properties were same as those in Example 1.

### Example 3

194.18 parts by weight of dimethyl terephthalate, 173.79 parts by weight of ethylene glycol, and 0.01 parts by weight of tetrabutyl titanate were reacted at about 200° C. for about 2 hours, and then heated to about 260° C. and vacuumed to a pressured of about 4 torr to react for about 1 hour, and then heated to about 270° C. and vacuumed to a pressured of about 0.1 torr to react until its intrinsic viscosity achieving 0.502 dL/g. The product such as PET (intrinsic viscosity was 0.502 dL/g) serving as first polyester was put into a vacuum oven, heated to about 120° C. and vacuumed to remove water. Hydroxyapatite powder (original average diameter was about 60 nm) served as bio-compatible ceramic powder. 60 parts by weight of the anhydrous PET and 40 parts by weight of hydroxyapatite powder were fed into a twin screw extruder, and then melt blended and dispersed at a screw temperature of about 265° C. and a rotating speed of 40 rpm to prepare ceramic powder composition. The cytotoxicity of the ceramic powder composition was measured according to the standard ISO10993-1 (MTT assay), and its cell viability was 70% (non-cytotoxicity).

### Example 4

Example 4 was similar to Example 3, and the difference in Example 4 was the weight ratio of the first polyester and the hydroxyapatite being changed from 60:40 to 40:60. The other processes and method of measuring properties were same as those in Example 3.

### Example 5

Commercially available PET (T-2150T from Shinkong Synthetic Fibers Corp., intrinsic viscosity was 0.535 dL/g) serving as first polyester was put into a vacuum oven, heated to about 120° C. and vacuumed to remove water. Hydroxyapatite powder (original average diameter was about 60 nm) served as bio-compatible ceramic powder. 60 parts by weight of the anhydrous PET and 40 parts by weight of hydroxyapatite powder were fed into a twin screw extruder, and then melt blended and dispersed at a screw temperature of about 265° C. and a rotating speed of 40 rpm to prepare ceramic powder composition. The cytotoxicity of the ceramic powder composition was measured according to the standard ISO10993-1 (MTT assay), and its cell viability was 70% (non-cytotoxicity).

### Example 6

Example 6 was similar to Example 5, and the difference in Example 6 was the weight ratio of the first polyester and the hydroxyapatite being changed from 60:40 to 40:60. The other processes and method of measuring properties were same as those in Example 5.

TABLE 1

| | | Raw material amount | | |
|---|---|---|---|---|
| | First | First | | |
| | polyester | polyester | Hydroxyapatite | Cytotoxicity test |
| Example | IV(dL/g) | (wt %) | (wt %) | (Cell viability, %) |
| 1 | 0.433 | 60 | 40 | 80% (Pass) |
| 2 | | 40 | 60 | 82% (Pass) |
| 3 | 0.502 | 60 | 40 | 81% (Pass) |
| 4 | | 40 | 60 | 83% (Pass) |
| 5 | 0.535 | 60 | 40 | 82% (Pass) |
| 6 | | 40 | 60 | 83% (Pass) |

* Standard of passing cytotoxicity test: Cell viability ≥70%

### Example 7

Commercially available PET (C-0226C from Shinkong Synthetic Fibers Corp., intrinsic viscosity was 0.66 dL/g) serving as second polyester was put into a vacuum oven, heated to about 120° C. and vacuumed to remove water. 98.33 parts by weight of the anhydrous second polyester (PET) and 1.67 parts by weight of the ceramic powder composition in Example 4 were fed into a twin screw extruder, and then melt blended and dispersed at a screw temperature of about 270° C. and a rotating speed of 40 rpm to prepare a composite material. The intrinsic viscosity of the composite material was measured according to the standard ASTM D4603. The cytotoxicity of the composite material was measured according to the standard ISO10993-1 (MTT assay), and its cell viability was 70% (non-cytotoxicity).

### Example 8

Example 8 was similar to Example 7, and the difference in Example 8 was the weight ratio of the second polyester and the ceramic powder composition being changed from 98.33:1.67 to 96.67:3.33. The other processes and method of measuring properties were same as those in Example 7.

### Example 9

Example 9 was similar to Example 7, and the difference in Example 9 was the weight ratio of the second polyester and the ceramic powder composition being changed from 98.33:1.67 to 93.34:6.66. The other processes and method of measuring properties were same as those in Example 7.

TABLE 2

| | Raw material amount | | Composite material properties | | |
|---|---|---|---|---|---|
| | Ceramic powder | Second | Bio-compatible | | |
| | composition | polyester | ceramic | | Cytotoxicity test |
| Example | (Example 4) (wt %) | (wt %) | (wt %) | IV(dL/g) | (Cell viability, %) |
| 7 | 1.67 | 98.33 | 1 | 0.636 | 87% (Pass) |
| 8 | 3.33 | 96.67 | 2 | 0.633 | 88% (Pass) |
| 9 | 6.66 | 93.34 | 4 | 0.629 | 89% (Pass) |

* Standard of passing cytotoxicity test: Cell viability ≥70%

### Example 10

Commercially available PET (C-0226C from Shinkong Synthetic Fibers Corp., intrinsic viscosity was 0.66 dL/g) serving as second polyester was put into a vacuum oven, heated to about 120° C. and vacuumed to remove water. 97.5 parts by weight of the anhydrous second polyester (PET) and 2.5 parts by weight of the ceramic powder composition in Example 1 were fed into a twin screw extruder, and then melt blended and dispersed at a screw temperature of about 270° C. and a rotating speed of 40 rpm to prepare a composite material. The intrinsic viscosity of the composite material was measured according to the standard ASTM D4603. The cytotoxicity of the composite material was measured according to the standard ISO10993-1 (MTT assay), and its cell viability was 70% (non-cytotoxicity).

### Example 11

Example 11 was similar to Example 10, and the difference in Example 11 was the ceramic powder composition in Example 1 being replaced with the ceramic powder composition in Example 3. The other processes and method of measuring properties were same as those in Example 10.

### Example 12

Example 12 was similar to Example 10, and the difference in Example 12 was the ceramic powder composition in Example 1 being replaced with the ceramic powder composition in Example 5. The other processes and method of measuring properties were same as those in Example 10.

TABLE 3

| | Raw material | | Composite material properties | | |
|---|---|---|---|---|---|
| | Ceramic powder | Second | Bio-compatible | | |
| | composition | polyester | ceramic | | Cytotoxicity test |
| Example | Amount (wt %) | (wt %) | powder (wt %) | IV(dL/g) | (Cell viability, %) |
| 10 | 2.5 (Example 1) | 97.5 | 0.83 | 0.621 | 87% (Pass) |
| 11 | 2.5 (Example 3) | 97.5 | 0.81 | 0.625 | 86% (Pass) |
| 12 | 2.5 (Example 5) | 97.5 | 0.86 | 0.634 | 87% (Pass) |

* Standard of passing cytotoxicity test: Cell viability ≥70%

### Example 13

The composite material in Example 8 was spun by melt spinning. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. The stretching ratio was 3.4%. The fiber had a fineness of 8.1 den, strength of 3.4±0.5 g/den, and an elongation of 20.6%. The cytotoxicity of the fiber was measured according to the standard ISO10993-1 (MTT assay), and its cell viability was 70% (non-cytotoxicity). In addition, the cell viability of the fiber prepared from the composite material was >100%, which means the composite material in the fiber manner could promote the cell growth.

### Example 14

Example 14 was similar to Example 13, and the difference in Example 14 was the stretching ratio of the fiber being changed from 3.4% to 3.8%. The other processes and method of measuring properties were same as those in Example 14.

TABLE 4

| | | | | | | | Cytotoxicity |
|---|---|---|---|---|---|---|---|
| | Composite | Throughput | Stretching | Fineness | Strength | Elongation | test (Cell |
| Example | material | (g/min) | ratio (%) | (den) | (g/den) | (%) | viability, %) |
| 13 | Example 8 | 0.26 | 3.4 | 8.1 | 3.4 ± 0.5 | 20.6 | 107% |
| | | | | | | | (Pass) |
| 14 | | 0.26 | 3.8 | 7.6 | 3.8 ± 0.4 | 21.2 | 106% |
| | | | | | | | (Pass) |

* Standard of passing cytotoxicity test: Cell viability ≥70%

As shown in Table 4, the fibers in some Examples had a tensile strength of about 2.5 g/den to 5.5 g/den.

### Comparative Example 1

Comparative Example 1 was similar to Example 13, and the difference in Comparative Example 1 was the composite material being replaced with PET (C-0226C commercially available from Shinkong Synthetic Fibers Corporation.). After melt spinning the PET fiber, the cell T2B004 P5 was used to perform cell culture attachment and bone differentiation test for measuring the sign of important differentiation (RUNX2) of the PET fiber. However, the pure PET fiber (without the bio-compatible ceramic powder dispersed therein) had no effect of promoting the cell bone differentiation.

### Example 15

The cell T2B004 P5 was used to perform cell culture attachment and bone differentiation test for measuring the sign of important differentiation (RUNX2) of the fiber in Example 13. The bone differentiation of the composite material fiber was 5 times faster than the pure PET fiber, and the cell attachment of the composite material fiber was also excellent.

TABLE 5

| | | | | Performance of promoting cell bone | | | |
|---|---|---|---|---|---|---|---|
| | Material for | | | differentiation (RUNX2) | | | |
| | test | Cell | Cell density | 7 days | 14 days | 21 days | 28 days |
| Comparative | Pure PET | T2B004 P5 | 2.0E4/ | 1 | 1.09 | 0.6 | 0.96 |
| Example 1 | | | 40 ul-piece | | | | |
| Example 15 | Composite | | | 1 | 1.10 | 1.42 | 5.18 |
| | material of | | | | | | |
| | Example 8 | | | | | | |

### Comparative Example 2

Commercially available PET (C-0226C from Shinkong Synthetic Fibers Corp., intrinsic viscosity was 0.66 dL/g) serving as polyester was put into a vacuum oven, heated to about 120° C. and vacuumed to remove water. Hydroxyapatite powder (original average diameter was about 60 nm) served as bio-compatible ceramic powder. 98 parts by weight of the anhydrous polyester and 2 parts by weight of the hydroxyapatite powder were fed into a twin screw extruder, and then melt blended and dispersed at a screw temperature of about 270° C. and a rotating speed of 40 rpm to prepare a composite material. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. However, the ceramic powder in the composite material seriously aggregated to block the spinning nozzle and break filament. The fibers in Comparative Example 2 and Example 8 were compared and analyzed by a scanning electron microscope (SEM), and the diameter distributions of the bio-compatible ceramic powder regions in the fibers are tabulated as below:

TABLE 6

| | | | Diameter of the bio-compatible ceramic powder | | | | | |
|---|---|---|---|---|---|---|---|---|
| | Hydroxyapatite | PET | regions (nm) | | | | | Spinning |
| | (wt %) | (wt %) | 50-200 | 200-300 | 300-400 | 400-500 | >500 | ability |
| Comparative | 2 | 98 | 50% | 37% | 8% | 2% | 4% | Blocking |
| Example 2 | | | | | | | | the |
| | | | | | | | | spinning |
| | | | | | | | | nozzle to |
| | | | | | | | | break |
| | | | | | | | | filament |
| Example 8 | 2 | 98 | 87% | 10% | 2% | 1% | 0% | Smooth |
| | | | | | | | | spinning |

As shown in Table 6, the bio-compatible ceramic powder was not pre-dispersed by the first polyester and directly dispersed in the second polyester in Comparative Example 2 would cause the powder aggregation. In the disclosure, the bio-compatible ceramic powder was firstly dispersed in the first polyester with a lower intrinsic viscosity to form a ceramic powder composition, and the ceramic powder composition was then dispersed in the second polyester with a higher intrinsic viscosity for reducing the aggregation degree of the bio-compatible ceramic powder. For example, more than 90% or even more than 95% of the bio-compatible ceramic powder regions had a diameter of less than or equal to 300 nm.

### Comparative Example 3

Commercially available PET (PCG60 from SABIC, intrinsic viscosity was 0.60 dL/g) serving as first polyester was put into a vacuum oven, heated to about 120° C. and vacuumed to remove water. Hydroxyapatite powder (original average diameter was about 60 nm) served as bio-compatible ceramic powder. 60 parts by weight of the anhydrous PET and 40 parts by weight of hydroxyapatite powder were fed into a twin screw extruder, and then melt blended and dispersed at a screw temperature of about 265° C. and a rotating speed of 40 rpm to prepare ceramic powder composition. Commercially available PET (C-0226C from Shinkong Synthetic Fibers Corp., intrinsic viscosity was 0.66 dL/g) serving as second polyester was put into a vacuum oven, heated to about 120° C. and vacuumed to remove water. 97.5 parts by weight of the anhydrous second polyester (PET) and 2.5 parts by weight of the ceramic powder composition were fed into a twin screw extruder, and then melt blended and dispersed at a screw temperature of about 270° C. and a rotating speed of 40 rpm to prepare a composite material. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. However, the ceramic powder in the composite material seriously aggregated to block the spinning nozzle and break filament.

### Example 16

The composite material in Example 11 was spun by melt spinning. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. The stretching ratio was 3.4%.

### Example 17

The composite material in Example 12 was spun by melt spinning. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. The stretching ratio was 3.4%.

TABLE 7

| | Polyester composition | | | | | | |
|---|---|---|---|---|---|---|---|
| | First | Second | | Fiber composition | | | Fiber |
| | polyester | polyester | ΔIV | Hydroxyapatite | PET | Spinning | diameter |
| | IV (dL/g) | IV (dL/g) | (dL/g) | (wt %) | (wt %) | ability | (μm) |
| Comparative | 0.60 | 0.66 | 0.06 | 1 | 99 | Blocking the | — |
| Example 3 | | | | | | spinning | |
| | | | | | | nozzle to | |
| | | | | | | break filament | |
| Example 16 | 0.502 | 0.66 | 0.158 | 1 | 99 | Smooth | 25.9 ± 1.4 |
| (Composite | | | | | | spinning | |
| material of | | | | | | | |
| Example 11) | | | | | | | |
| Example 17 | 0.535 | 0.66 | 0.125 | 1 | 99 | Smooth | 26.1 ± 1.2 |
| (Composite | | | | | | spinning | |
| material of | | | | | | | |
| Example 12) | | | | | | | |

As shown in Table 7, the ΔIV less than 0.1 dL/g in Comparative Example 3 would result in poor powder dispersion, thereby blocking the spinning nozzle to break filament. In the disclosure, the bio-compatible ceramic powder was firstly dispersed in the first polyester with a lower intrinsic viscosity to form a ceramic powder composition, and the ceramic powder composition was then dispersed in the second polyester with a higher intrinsic viscosity, in which ΔIV of the first polyester and the second polyester was greater than or equal to 0.1 could reduce the aggregation degree of the bio-compatible ceramic powder.

### Comparative Example 4

1 part by weight of hydroxyapatite powder (original average diameter was 60 nm) serving as bio-compatible ceramic powder, 0.67 parts by weight of dispersant A (Solplus™ DP320, commercially available from Lubrizol Advanced Materials, Inc.), and commercially available PET (C-0226C from Shinkong Synthetic Fibers Corp., intrinsic viscosity was 0.66 dL/g) serving as second polyester were fed into a twin screw extruder, and then melt blended and dispersed at a screw temperature of about 270° C. and a rotating speed of 40 rpm to prepare composite material. The cytotoxicity of the composite material was measured according to the standard ISO10993-1 (MTT assay), and its cell viability was <70% (cytotoxicity).

### Comparative Example 5

Comparative Example 5 was similar to Comparative Example 4, and the difference in Comparative Example 5 was the dispersant A being replaced with a dispersant B (BYK P4102 commercially available from BYK). The other processes and method of measuring properties were same as those in Comparative Example 4.

### Comparative Example 6

Comparative Example 6 was similar to Comparative Example 4, and the difference in Comparative Example 6 was the dispersant A being replaced with a dispersant C (DISPERPLAST-1018 commercially available from BYK). The other processes and method of measuring properties were same as those in Comparative Example 4.

TABLE 8

| | Bio-compatible | First | | | Second | |
|---|---|---|---|---|---|---|
| | ceramic | polyester | | | polyester | Cytotoxicity test |
| | (%) | (%) | Dispersant (%) | | (wt %) | (Cell viability, %) |
| Example 7 | 1 | 0.67 | — | — | 98.33 | 87% |
| | | | | | | (Pass) |
| Comparative | 1 | — | Dispersant A | 0.67 | 98.33 | 45% |
| Example 4 | | | | | | (Not Pass) |
| Comparative | 1 | — | Dispersant B | 0.67 | 98.33 | 37% |
| Example 5 | | | | | | (Not Pass) |
| Comparative | 1 | — | Dispersant C | 0.67 | 98.33 | 25% |
| Example 6 | | | | | | (Not Pass) |

* Standard of passing cytotoxicity test: Cell viability ≥70%

As shown in Table 8, the composite material utilizing the small molecular dispersant was improper to be applied as medical materials (e.g. artificial ligament/tendon) due to its cytotoxicity.

### Comparative Example 7

98.98 parts by weight of anhydrous PET (C-0226C commercially available from Shinkong Synthetic Fibers Corp., intrinsic viscosity was 0.66 dL/g) serving as first polyester and 1.02 parts by weight of hydroxyapatite powder (original average diameter was about 60 nm) serving as bio-compatible ceramic powder were fed into a twin screw extruder, and then melt blended and dispersed at a screw temperature of about 265° C. and a rotating speed of 40 rpm to prepare ceramic powder composition. 1.96 parts by weight of anhydrous PET (T-2150T commercially available from Shinkong Synthetic Fibers Corp., intrinsic viscosity was 0.535 dL/g) serving as second polyester and 98.04 parts by weight of the ceramic powder composition were fed into a twin screw extruder, and then melt blended and dispersed at a screw temperature of about 270° C. and a rotating speed of 40 rpm to prepare a composite material. The composite material was fed into a screw extruder, sent to a heating zone by a rotating screw, then sent to a metering pump after melting and extrusion for being spun at a spinning temperature of 290° C. and a spinning speed of 64 m/min, and then stretched at 110° C. to form a fiber. However, the ceramic powder in the composite material seriously aggregated to block the spinning nozzle and break filament.

TABLE 9

| | | Fiber composition | | |
|---|---|---|---|---|
| | | Hydroxyapatite | PET | Spinning |
| | Adding order | (wt %) | (wt %) | ability |
| Comparative | Firstly adding the | 1 | 99 | Blocking the |
| Example 7 | polyester with | | | spinning |
| | high IV, and then | | | nozzle to |
| | adding the | | | break |
| | polyester with | | | filament |
| | low IV | | | |
| Example 17 | Firstly adding the | 1 | 99 | Smooth |
| (Composite | polyester with | | | spinning |
| material in | low IV, and then | | | |
| Example 12) | adding the | | | |
| | polyester with | | | |
| | high IV | | | |

As shown in Table 9, the reverse order (firstly adding the polyester with high IV, and then adding the polyester with low IV) would result in a poor powder dispersion to block the spinning nozzle and break filament. In the disclosure, the bio-compatible ceramic powder was firstly dispersed in the first polyester with a lower intrinsic viscosity to form a ceramic powder composition, and the ceramic powder composition was then dispersed in the second polyester with a higher intrinsic viscosity for reducing the aggregation degree of the bio-compatible ceramic powder.

### Comparative Example 8

97.5 parts by weight of anhydrous PET (C-0226C commercially available from Shinkong Synthetic Fibers Corp., intrinsic viscosity was 0.66 dL/g), 1.5 parts by weight of anhydrous PET (T-2150T commercially available from Shinkong Synthetic Fibers Corp., intrinsic viscosity was 0.535 dL/g), and 1 part by weight of hydroxyapatite powder (original average diameter was about 60 nm) were simultaneously fed into a twin screw extruder, and then melt blended and dispersed at a screw temperature of about 270° C. and a rotating speed of 40 rpm to prepare a composite material. The composite material was fed into a screw extruder, sent to a heating zone by a rotating screw, then sent to a metering pump after melting and extrusion for being spun at a spinning temperature of 290° C. and a spinning speed of 64 m/min. However, the ceramic powder in the composite material seriously aggregated to block the spinning nozzle and break filament.

Artificial Ligament: Clinical Animal Efficacy Verification

New Zealand white rabbits (about 3 kg) were selected as experimental animals to perform ligament reconstruction surgery on medial collateral ligament (MCL) of the rabbits. The experiments were classified as two groups: (1) Comparative Example 9: an artificial ligament commercially available from Orthomed (pure PET), and (2) Example 18: the fiber in Example 8 was woven by plane weaving to form an artificial ligament. The rabbits were anesthetized by Zoletil50 and Rompun 20 (1:1, 0.5 mL/kg) before surgical operation, and the hind knee joint was opened after the operation. A skin incision was made along the anterolateral side of the knee joint and the lateral side of the patella, and the synovial sac of the knee joint was opened through the incision. Two groups of the artificial ligaments were respectively implanted next to the autologous MCL (with a small cut) and sewn with the MCL. Thereafter, the opened tissue and skin were sutured to complete the operation. The animal care was made after the operation. Nine ligament reconstruction surgeries on MCL were performed for each group, and the effect in 1 month, 3 months, and 6 months after the operation were evaluated.

The alanine aminotransferase (ALT), creatinine, and blood urea nitrogen (BUN) of the rabbits in 0 month, 1 month, and 3 months after implanting the artificial ligament were all within the normal reference value range (ALT: 22-80 iu/litre, BUN 17-24 mg/dL, creatinine: 0.8-1.8 mg/dL), Accordingly, the artificial ligaments did not cause liver or kidney toxicity after being surgically implanted into animals, which means that they were bio-compatible.

The artificial ligaments of each group were sampled in one month and three months after being surgically implanted into the animals. The artificial ligaments and the bone tissues connected to the front and back ends of the artificial ligaments were taken out. Observation from eye shows that in Example 18 and Comparative Example 9, both the artificial ligament and the bone nail were clearly visible in one month after the operation, and the artificial ligament and the bone nail were covered by soft tissue and invisible in three months after the operation. After the artificial ligament was taken out, it was also found that the surrounding soft tissue successfully grew into the artificial ligament as the ligamentation phenomenon.

According to the X-ray images of the artificial ligament surgically implanted into the rabbit in one month and three months of each group, parts of the artificial ligament was loosened and a gap was produced between the ligament buckle and the bone in Example 18 and Comparative Example 9. According to the X-ray images in three months after the operation, there was healing phenomenon between the bone nail and the bone drill.

The average ultimate tensile strength of the artificial ligament after being surgically implanted into the rabbit for 1 month in Example 18 was about 100 N, and the average ultimate tensile strength of the artificial ligament after being surgically implanted into the rabbit for 1 month in Comparative Example 9 was about 60 N. The fiber in Example 9 had a better effect to promote cellular bone differentiation than the pure PET, which is also one factor that influenced the ultimate tensile strength of Example 18 better than that of Comparative Example 9.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A fiber, comprising:
0.5 to 4 parts by weight of a bio-compatible ceramic powder region; and
96 to 99.5 parts by weight of a polyester region,
wherein the bio-compatible ceramic powder region is distributed in the polyester region, at least 90% of the bio-compatible ceramic powder region has a diameter of less than or equal to 300 nm and greater than 10 nm, and the cell viability of bio-toxicity test of the fiber is higher than 70%.

2. The fiber as claimed in claim 1, having a diameter of 2 micrometers to 150 micrometers.

3. The fiber as claimed in claim 1, wherein the polyester region comprises polyethylene terephthalate, polybutylene terephthalate, or a combination thereof, and the bio-compatible ceramic powder region comprises hydroxyapatite, tricalcium phosphate, calcium sulfate, or a combination thereof.

4. The fiber as claimed in claim 1, being free of dispersant.

5. The fiber as claimed in claim 1, having a cell viability of bio-toxicity test higher than 100%.

6. An artificial ligament/tendon, being woven from the fiber as claimed in claim 1.

7. A method of preparing a fiber, comprising:
blending bio-compatible ceramic powder and first polyester to form a ceramic powder composition, wherein the bio-compatible ceramic powder and the first polyester have a weight ratio of 10:90 to 60:40;
blending the ceramic powder composition and second polyester to form a composite material, wherein the ceramic powder composition and the second polyester have a weight ratio of 0.4:99.6 to 40:60; and
spinning the composite material to form a fiber,
wherein the first polyester has an intrinsic viscosity (IV) of 0.35 dL/g to 0.55 dL/g, and the second polyester has an intrinsic viscosity (IV) of 0.6 dL/g to 0.8 g/dL.

8. The method as claimed in claim 7, wherein the fiber comprises:
0.5 to 4 parts by weight of a bio-compatible ceramic powder region; and
96 to 99.5 parts by weight of a polyester region,
wherein the bio-compatible ceramic powder region is distributed in the polyester region, at least 90% of the bio-compatible ceramic powder region has a diameter of less than or equal to 300 nm and greater than 10 nm, and the cell viability of bio-toxicity test of the fiber is higher than 70%.

9. The method as claimed in claim 7, wherein the fiber has a diameter of 2 micrometers to 150 micrometers.

10. The method as claimed in claim 1, wherein the first polyester and the second polyester comprise polyethylene terephthalate, polybutylene terephthalate, or a combination thereof, and the bio-compatible ceramic powder comprises hydroxyapatite, tricalcium phosphate, calcium sulfate, or a combination thereof.

11. The method as claimed in claim 7, wherein the fiber is free of dispersant.

12. The method as claimed in claim 7, wherein the intrinsic viscosity difference (ΔIV) between the first polyester and the second polyester is greater than or equal to 0.1 dL/g and less than or equal to 0.45 dL/g.
